(19) United States
(12) Patent Application Publication
(10) Pub. No.: US 2019/0279767 A1
(43) Pub. Date: Sep. 12, 2019

(54) SYSTEMS AND METHODS FOR CREATING AN EXPERT-TRAINED DATA MODEL

(71) Applicant: James Stewart Bates, Paradise Valley, AZ (US)

(72) Inventor: James Stewart Bates, Paradise Valley, AZ (US)

(21) Appl. No.: 15/913,780

(22) Filed: Mar. 6, 2018

(51) Int. Cl. G16H 50/20; G06F 17/27; G06F 17/30; G06K 9/62

(52) U.S. Cl. CPC G16H 50/20; G06F 17/2705; G06K 9/6256; G06F 17/30719; G06F 17/30616

(57) ABSTRACT

Presented are systems and methods for using expert knowledge to generate, train, and use a medical data model that uses medical data from a number of sources to generate likelihoods that a given set of symptoms is caused or related to one or more illnesses. Various embodiments accomplish this by parsing medical and non-medical data into keywords and target words to learn, e.g., based on a characteristic of the parsed words, an association between keywords and target words. Based on the learned associations, likelihood scores are then generated that represent, for example, a relationship between a set of symptoms and an illness, a treatment, and an outcome.

### BACKGROUND

### Technical Field

The present disclosure relates to health care, and more particularly, to systems and methods for providing doctors and patients with treatment suggestions based on computer-aided diagnoses.

### Background of the Invention

Patients' common problems with scheduling an appointment with a primary doctor when needed or in a time-efficient manner is causing a gradual shift away from patients establishing and relying on a life-long relationship with a single general practitioner, who diagnoses and treats a patient in health-related matters, towards patients opting to receive readily available treatment in urgent care facilities that are located near home, work, or school and provide relatively easy access to health care without the inconvenience of appointments that oftentimes must be scheduled weeks or months ahead of time. Yet, the decreasing importance of primary doctors makes it difficult for different treating physicians to maintain a reasonably complete medical record for each patient, which results in a patient having to repeat a great amount of information personal and medical each time when visiting a different facility or different doctor. In some cases, patients confronted with lengthy and time-consuming patient questionnaires fail to provide accurate information that may be important for a proper medical treatment, whether for the sake of expediting their visit or other reasons. In addition, studies have shown that patients attending urgent care or emergency facilities may in fact worsen their health conditions due to the risk of exposure to bacteria or viruses in medical facilities despite the medical profession's efforts to minimize the number of such instances.

Through consistent regulation changes, electronic health record changes and pressure from payers, both health care facilities and providers are looking for ways to make patient intake, triage, diagnosis, treatment, electronic health record data entry, treatment, billing, and patient follow-up activity more efficient, provide better patient experience, and increase the doctor to patient throughput per hour, while simultaneously reducing cost.

The desire to increase access to health care providers, a pressing need to reduce health care costs in developed countries and the goal of making health care available to a larger population in less developed countries have fueled the idea of telemedicine. In most cases, however, video or audio conferencing with a doctor does not provide sufficient patient-physician interaction that is necessary to allow for a proper medical diagnosis to efficiently serve patients.

What is needed are systems and methods that ensure reliable remote or local medical patient intake, triage, diagnosis, treatment, electronic health record data entry/management, treatment, billing and patient follow-up activity so that physicians can allocate patient time more efficiently and, in some instances, allow individuals to manage their own health, thereby, reducing health care costs.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 illustrates an exemplary diagnostic system according to embodiments of the present disclosure.

FIG. 2 illustrates an exemplary medical instrument equipment system according to embodiments of the present disclosure.

FIG. 3 illustrates an exemplary medical data system according to embodiments of the present disclosure.

FIG. 4 is a flowchart illustrating a process for training a medical data model according to various embodiments of the present disclosure.

FIG. 5 is a flowchart illustrating a process for using a medical data model, according to various embodiments of the present disclosure, to make medical predictions.

FIG. 6 is a flowchart illustrating a process for using expert knowledge to create and/or train a medical data model according to various embodiments of the present disclosure.

FIG. 7 depicts a simplified block diagram of a computing device/information handling system according to embodiments of the present disclosure.

### DETAILED DESCRIPTION OF THE PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Elements/components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components/elements. Components/elements may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. Also, additional or fewer connections may be used. It shall also be noted that the terms “coupled” “connected” or “communicatively coupled” shall be understood to include direct connections, indirect connections through one or more intermediary devices, and wireless connections.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims.

Furthermore, the use of certain terms in various places in the specification is for illustration and should not be construed as limiting. A service, function, or resource is not limited to a single service, function, or resource; usage of these terms may refer to a grouping of related services, functions, or resources, which may be distributed or aggregated.

In this document, the term “sensor” refers to a device capable of acquiring information related to any type of physiological condition or activity (e.g., a biometric diagnostic sensor); physical data (e.g., a weight); and environmental information (e.g., ambient temperature sensor), including hardware-specific information. The term “position” refers to spatial and temporal data (e.g., orientation and motion information). “Doctor” refers to any health care professional, health care provider, physician, or person directed by a physician. “Patient” is any user who uses the systems and methods of the present invention, e.g., a person being examined or anyone assisting such person. The term illness may be used interchangeably with the term diagnosis. As used herein, “answer” or “question” refers to one or more of 1) an answer to a question, 2) a measurement or measurement request (e.g., a measurement performed by a “patient”), and 3) a symptom (e.g., a symptom selected by a “patient”).

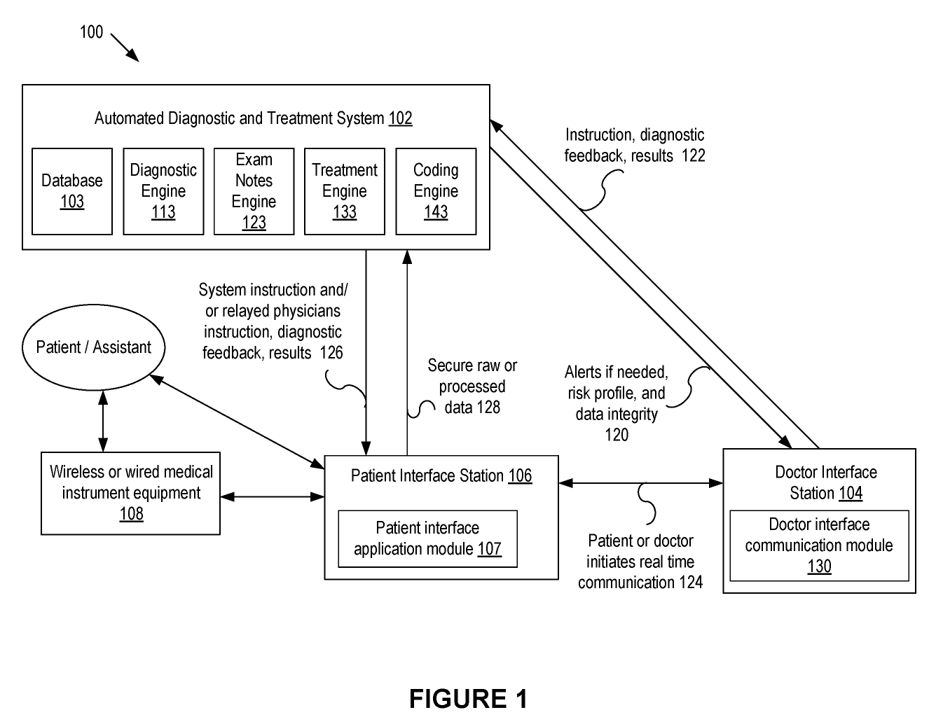

FIG. 1 illustrates an exemplary diagnostic system according to embodiments of the present disclosure. Diagnostic system 100 comprises automated diagnostic and treatment system 102, patient interface station 106, doctor interface station 104, and medical instrument equipment 108. Automated diagnostic and treatment system 102 may further comprise database 103, diagnostic engine 113, exam notes engine 123, treatment engine 133, and coding engine 143. Both patient interface station 106 and doctor interface station 104 may be implemented into any tablet, computer, mobile device, or other electronic device. Medical instrument equipment 108 is designed to collect mainly diagnostic patient data, and may comprise one or more diagnostic devices, for example, in a home diagnostic medical kit that generates diagnostic data based on physical and non-physical characteristics of a patient. It is noted that diagnostic system 100 may comprise additional sensors and devices that, in operation, collect, process, or transmit characteristic information about the patient, medical instrument usage, orientation, environmental parameters such as ambient temperature, humidity, location, and other useful information that may be used to accomplish the objectives of the present invention.

In operation, a patient may enter patient-related data, such as health history, patient characteristics, symptoms, health concerns, medical instrument measured diagnostic data, images, and sound patterns, or other relevant information into patient interface station 106. The patient may use any means of communication, such as voice control, to enter data, e.g., in the form of a questionnaire. Patient interface station 106 may provide the data raw or in processed form to automated diagnostic and treatment system 102, e.g., via a secure communication.

In embodiments, the patient may be prompted, e.g., by a software application, to answer questions intended to aid in the diagnosis of one or more medical conditions. The software application may provide guidance by describing how to use medical instrument equipment 108 to administer a diagnostic test or how to make diagnostic measurements for any particular device that may be part of medical instrument equipment 108 so as to facilitate accurate measurements of patient diagnostic data.

In embodiments, the patient may use medical instrument equipment 108 to create a patient health profile that serves as a baseline profile. Gathered patient-related data may be securely stored in database 103 or a secure remote server (not shown) coupled to automated diagnostic and treatment system 102. In embodiments, automated diagnostic and treatment system 102 enables interaction between a patient and a remotely located health care professional, who may provide instructions to the patient, e.g., by communicating via the software application. A doctor may log into a cloud-based system (not shown) to access patient-related data via doctor interface station 104. In embodiments, automated diagnostic and treatment system 102 presents automated diagnostic suggestions to a doctor, who may verify or modify the suggested information.

In embodiments, based on one more patient questionnaires, data gathered by medical instrument equipment 108, patient feedback, and historic diagnostic information, the patient may be provided with instructions, feedback, results 122, and other information pertinent to the patient's health. In embodiments, the doctor may select an illness based on automated diagnostic system suggestions and/or follow a sequence of instructions, feedback, and/or results 122 may be adjusted based on decision vectors associated with a medical database. In embodiments, medical instrument equipment 108 uses the decision vectors to generate a diagnostic result, e.g., in response to patient answers and/or measurements of the patient's vital signs.

In embodiments, medical instrument equipment 108 comprises a number of sensors, such as accelerometers, gyroscopes, pressure sensors, cameras, bolometers, altimeters, IR LEDs, and proximity sensors that may be coupled to one or more medical devices, e.g., a thermometer, to assist in performing diagnostic measurements and/or monitor a patient's use of medical instrument equipment 108 for accuracy. A camera, bolometer, or other spectrum imaging device (e.g. radar), in addition to taking pictures of the patient, may use image or facial recognition software and machine vision to recognize body parts, items, and actions to aid the patient in locating suitable positions for taking a measurement on the patient's body, e.g., by identifying any part of the patient's body as a reference.

Examples of the types of diagnostic data that medical instrument equipment 108 may generate comprise body temperature, blood pressure, images, sound, heart rate, blood oxygen level, motion, ultrasound, pressure or gas analysis, continuous positive airway pressure, electrocardiogram, electroencephalogram, Electrocardiography, BMI, muscle mass, blood, urine, and any other patient-related data 128. In embodiments, patient-related data 128 may be derived from a non-surgical wearable or implantable monitoring device that gathers sample data.

In embodiments, an IR LED, proximity beacon, or other identifiable marker (not shown) may be attached to medical instrument equipment 108 to track the position and placement of medical instrument equipment 108. In embodiments, a camera, bolometer, or other spectrum imaging device uses the identifiable marker as a control tool to aid the camera or the patient in determining the position of medical instrument equipment 108.

In embodiments, machine vision software may be used to track and overlay or superimpose, e.g., on a screen, the position of the identifiable marker e.g., IR LED, heat source, or reflective material with a desired target location at which the patient should place medical instrument equipment 108, thereby, aiding the patient to properly place or align a sensor and ensure accurate and reliable readings. Once medical instrument equipment 108, e.g., a stethoscope is placed at the desired target location on a patient's torso, the patient may be prompted by optical or visual cues to breath according to instructions or perform other actions to facilitate medical measurements and to start a measurement.

In embodiments, one or more sensors that may be attached to medical instrument equipment 108 monitor the placement and usage of medical instrument equipment 108 by periodically or continuously recording data and comparing measured data, such as location, movement, and angles, to an expected data model and/or an error threshold to ensure measurement accuracy. A patient may be instructed to adjust an angle, location, or motion of medical instrument equipment 108, e.g., to adjust its state and, thus, avoid low-accuracy or faulty measurement readings. In embodiments, sensors attached or tracking medical instrument equipment 108 may generate sensor data and patient interaction activity data that may be compared, for example, against an idealized patient medical instrument equipment usage sensor model data to create an equipment usage accuracy score. The patient medical instrument equipment measured medical data may also be compared with idealized device measurement data expected from medical instrument equipment 108 to create a device accuracy score.

Feedback from medical instrument equipment 108 (e.g., sensors, proximity, camera, etc.) and actual device measurement data may be used to instruct the patient to properly align medical instrument equipment 108 during a measurement. In embodiments, medical instrument equipment type and sensor system monitoring of medical instrument equipment 108 patient interaction may be used to create a device usage accuracy score for use in a medical diagnosis algorithm. Similarly, patient medical instrument equipment measured medical data may be used to create a measurement accuracy score for use by the medical diagnostic algorithm.

In embodiments, machine vision software may be used to show on a monitor an animation that mimics a patient's movements and provides detailed interactive instructions and real-time feedback to the patient. This aids the patient in correctly positioning and operating medical instrument equipment 108 relative to the patient's body so as to ensure a high level of accuracy when using medical instrument equipment 108 is operated.

In embodiments, once automated diagnostic and treatment system 102 detects unexpected data, e.g., data representing an unwanted movement, location, measurement data, etc., a validation process comprising a calculation of a trustworthiness score or reliability factor is initiated in order to gauge the measurement accuracy. Once the accuracy of the measured data falls below a desired level, the patient may be asked to either repeat a measurement or request assistance by an assistant, who may answer questions, e.g., remotely via an application to help with proper equipment usage, or alert a nearby person to assist with using medical instrument equipment 108. The validation process, may also instruct a patient to answer additional questions, and may comprise calculating the measurement accuracy score based on a measurement or re-measurement.

In embodiments, upon request 124, automated diagnostic and treatment system 102 may enable a patient-doctor interaction by granting the patient and doctor access to diagnostic system 100. The patient may enter data, take measurements, and submit images and audio files or any other information to the application or web portal. The doctor may access that information, for example, to review a diagnosis generated by automated diagnostic and treatment system 102, and generate, confirm, or modify instructions for the patient. Patient-doctor interaction, while not required for diagnostic and treatment, if used, may occur in person, real-time via an audio/video application, or by any other means of communication.

In embodiments, automated diagnostic and treatment system 102 may utilize images generated from a diagnostic examination of mouth, throat, eyes, ears, skin, extremities, surface abnormalities, internal imaging sources, and other suitable images and/or audio data generated from diagnostic examination of heart, lungs, abdomen, chest, joint motion, voice, and any other audio data sources. Automated diagnostic and treatment system 102 may further utilize patient lab tests, medical images, or any other medical data. In embodiments, automated diagnostic and treatment system 102 enables medical examination of the patient, for example, using medical devices, e.g., ultrasound, in medical instrument equipment 108 to detect sprains, contusions, or fractures, and automatically provide diagnostic recommendations regarding a medical condition of the patient.

In embodiments, diagnosis comprises the use of medical database decision vectors that are at least partially based on the patient's self-measured (or assistant-measured) vitals or other measured medical data, the accuracy score of a measurement dataset, a usage accuracy score of a sensor attached to medical instrument equipment 108, a regional illness trend, and information used in generally accepted medical knowledge evaluations steps. The decision vectors and associated algorithms, which may be installed in automated diagnostic and treatment system 102, may utilize one or more-dimensional data, patient history, patient questionnaire feedback, and pattern recognition or pattern matching for classification using images and audio data. In embodiments, a medical device usage accuracy score generator (not shown) may be implemented within automated diagnostic and treatment system 102 and may utilize an error vector of any device in medical instrument equipment or attached sensors 108 to create the device usage accuracy score and utilize the actual patient-measured device data to create the measurement data accuracy score.

In embodiments, automated diagnostic and treatment system 102 outputs diagnosis and/or treatment information that may be communicated to the patient, for example, electronically or in person by a medical professional, e.g., a treatment guideline that may include a prescription for a medication. In embodiments, prescriptions may be communicated directly to a pharmacy for pick-up or automated home delivery.

In embodiments, automated diagnostic and treatment system 102 may generate an overall health risk profile of the patient and recommend steps to reduce the risk of overlooking potentially dangerous conditions or guide the patient to a nearby facility that can treat the potentially dangerous condition. The health risk profile may assist a treating doctor in fulfilling duties to the patient, for example, to carefully review and evaluate the patient and, if deemed necessary, refer the patient to a specialist, initiate further testing, etc. The health risk profile advantageously reduces the potential for negligence and, thus, medical malpractice.

Automated diagnostic and treatment system 102, in embodiments, comprises a payment feature that uses patient identification information to access a database to, e.g., determine whether a patient has previously arranged a method of payment, and if the database does not indicate a previously arranged method of payment, automated diagnostic and treatment system 102 may prompt the patient to enter payment information, such as insurance, bank, or credit card information. Automated diagnostic and treatment system 102 may determine whether payment information is valid and automatically obtain an authorization from the insurance, EHR system, and/or the card issuer for payment for a certain amount for services rendered by the doctor. An invoice may be electronically presented to the patient, e.g., upon completion of a consultation, such that the patient can authorize payment of the invoice, e.g., via an electronic signature.

In embodiments, patient database 103 (e.g., a secured cloud-based database) may comprise a security interface (not shown) that allows secure access to a patient database, for example, by using patient identification information to obtain the patient's medical history. The interface may utilize biometric, bar code, or other electronically security methods. In embodiments, medical instrument equipment 108 uses unique identifiers that are used as a control tool for measurement data. Database 103 may be a repository for any type of data created, modified, or received by diagnostic system 100, such as generated diagnostic information, information received from patient's wearable electronic devices, remote video/audio data and instructions, e.g., instructions received from a remote location or from the application.

In embodiments, fields in the patient's electronic health care record (EHR) are automatically populated based on one or more of questions asked by diagnostic system 100, measurements taken by the patient/system 100, diagnosis and treatment codes generated by system 100, one or more trust scores, and imported patient health care data from one or more sources, such as an existing health care database. It is understood the format of imported patient health care data may be converted to be compatible with the EHR format of system 100. Conversely, exported patient health care data may be converted, e.g., to be compatible with an external EHR database.

In addition, patient-related data documented by system 100 provide support for the code decision for the level of exam a doctor performs. Currently, for billing and reimbursement purposes, doctors have to choose one of any identified codes (e.g., ICD10 currently holds approximately 97,000 medical codes) to identify an illness and provide an additional code that identifies the level of physical exam/diagnosis performed on the patient (e.g., full body physical exam) based on an illness identified by the doctor.

In embodiments, patient answers are used to suggest to the doctor a level of exam that is supported by the identified illness, e.g., to ensure that the doctor does not perform unnecessary in-depth exams for minor illnesses or a treatment that may not be covered by the patient's insurance.

In embodiments, upon identifying a diagnosis, system 100 generates one or more recommendations/suggestions/options for a particular treatment. In embodiments, one or more treatment plans are generated that the doctor may discuss with the patient and decide on a suitable treatment. For example, one treatment plan may be tailored purely for effectiveness, another one may consider the cost of drugs. In embodiments, system 100 may generate a prescription or lab test request and consider factors, such as recent research results, available drugs and possible drug interactions, the patient's medical history, traits of the patient, family history, and any other factors that may affect treatment when providing treatment information. In embodiments, diagnosis and treatment databases may be continuously updated, e.g., by health care professionals, so that an optimal treatment may be administered to a particular patient, e.g., a patient identified as member of a certain risk group.

It is noted that sensors and measurement techniques may be advantageously combined to perform multiple functions using a reduced number of sensors. For example, an optical sensor may be used as a thermal sensor by utilizing IR technology to measure body temperature. It is further noted that some or all data collected by system 100 may be processed and analyzed directly within automated diagnostic and treatment system 102 or transmitted to an external reading device (not shown in FIG. 1) for further processing and analysis, e.g., to enable additional diagnostics.

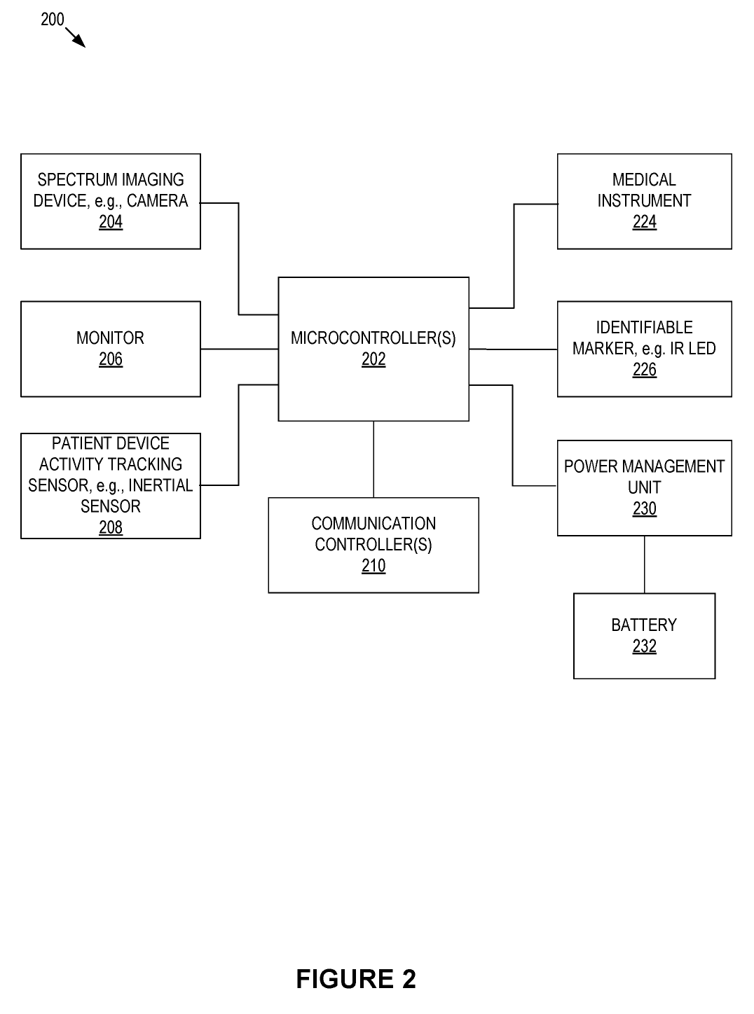

FIG. 2 illustrates an exemplary patient diagnostic measurement system according to embodiments of the present disclosure. As depicted, patient diagnostic measurement system 200 comprises microcontroller 202, spectrum imaging device, e.g., camera 204, monitor 206, patient-medical equipment activity tracking sensors, e.g., inertial sensor 208, communications controller 210, medical instruments 224, identifiable marker, e.g., IR LED 226, power management unit 230, and battery 232. Each component may be coupled directly or indirectly by electrical wiring, wirelessly, or optically to any other component in system 200.

Medical instrument 224 comprises one or more devices that are capable of measuring physical and non-physical characteristics of a patient that, in embodiments, may be customized, e.g., according to varying anatomies among patients, irregularities on a patient's skin, and the like. In embodiments, medical instrument 224 is a combination of diagnostic medical devices that generate diagnostic data based on patient characteristics. Exemplary diagnostic medical devices are heart rate sensors, otoscopes, digital stethoscopes, in-ear thermometers, blood oxygen sensors, high-definition cameras, spirometers, blood pressure meters, respiration sensors, skin resistance sensors, glucometers, ultrasound devices, electrocardiographic sensors, body fluid sample collectors, eye slit lamps, weight scales, and any devices known in the art that may aid in performing a medical diagnosis. In embodiments, patient characteristics and vital signs data may be received from and/or compared against wearable or implantable monitoring devices that gather sample data, e.g., a fitness device that monitors physical activity.

One or more medical instruments 224 may be removably attachable directly to a patient's body, e.g., torso, via patches or electrodes that may use adhesion to provide good physical or electrical contact. In embodiments, medical instruments 224, e.g., a contact-less thermometer, may perform contact-less measurements some distance away from the patient's body.

In embodiments, microcontroller 202 may be a secure microcontroller that securely communicates information in encrypted form to ensure privacy and the authenticity of measured data and activity sensor and patient-equipment proximity information and other information in patient diagnostic measurement system 200. This may be accomplished by taking advantage of security features embedded in hardware of microcontroller 202 and/or software that enables security features during transit and storage of sensitive data. Each device in patient diagnostic measurement system 200 may have keys that handshake to perform authentication operations on a regular basis.

Spectrum imaging device camera 204 is any audio/video device that may capture patient images and sound at any frequency or image type. Monitor 206 is any screen or display device that may be coupled to camera, sensors and/or any part of system 200. Patient-equipment activity tracking inertial sensor 208 is any single or multi-dimensional sensor, such as an accelerometer, a multi-axis gyroscope, pressure sensor, and a magnetometer capable of providing position, motion, pressure on medical equipment or orientation data based on patient interaction. Patient-equipment activity tracking inertial sensor 208 may be attached to (removably or permanently) or embedded into medical instrument 224. Identifiable marker IR LED 226 represents any device, heat source, reflective material, proximity beacon, altimeter, etc., that may be used by microcontroller 202 as an identifiable marker. Like patient-equipment activity tracking inertial sensor 208, identifiable marker IR LED 226 may be reattacheable to or embedded into medical instrument 224.

In embodiments, communication controller 210 is a wireless communications controller attached either permanently or temporarily to medical instrument 224 or the patient's body to establish a bi-directional wireless communications link and transmit data, e.g., between sensors and microcontroller 202 using any wireless communication protocol known in the art, such as Bluetooth Low Energy, e.g., via an embedded antenna circuit that wirelessly communicates the data. One of ordinary skill in the art will appreciate that electromagnetic fields generated by such antenna circuit may be of any suitable type. In case of an RF field, the operating frequency may be located in the ISM frequency band, e.g., 13.56 MHz. In embodiments, data received by wireless communications controller 210 may be forwarded to a host device (not shown) that may run a software application.

In embodiments, power management unit 230 is coupled to microcontroller 202 to provide energy to, e.g., microcontroller 202 and communication controller 210. Battery 232 may be a back-up battery for power management unit 230 or a battery in any one of the devices in patient diagnostic measurement system 200. One of ordinary skill in the art will appreciate that one or more devices in system 200 may be operated from the same power source (e.g., battery 232) and perform more than one function at the same or different times. A person of skill in the art will also appreciate that one or more components, e.g., sensors 208, 226, may be integrated on a single chip/system, and that additional electronics, such as filtering elements, etc., may be implemented to support the functions of medical instrument equipment measurement or usage monitoring and tracking system 200 according to the objectives of the invention.

In operation, a patient may use medical instrument 224 to gather patient data based on physical and non-physical patient characteristics, e.g., vital signs data, images, sounds, and other information useful in the monitoring and diagnosis of a health-related condition. The patient data is processed by microcontroller 202 and may be stored in a database (not shown). In embodiments, the patient data may be used to establish baseline data for a patient health profile against which subsequent patient data may be compared.

In embodiments, patient data may be used to create, modify, or update EHR data. Gathered medical instrument equipment data, along with any other patient and sensor data, may be processed directly by patient diagnostic measurement system 200 or communicated to a remote location for analysis, e.g., to diagnose existing and expected health conditions to benefit from early detection and prevention of acute conditions or aid in the development of novel medical diagnostic methods.

In embodiments, medical instrument 224 is coupled to a number of sensors, such as patient-equipment tracking inertial sensor 208 and/or identifiable marker IR LED 226, that may monitor a position/orientation of medical instrument 224 relative to the patient's body when a medical equipment measurement is taken. In embodiments, sensor data generated by sensor 208, 226 or other sensors may be used in connection with, e.g., data generated by spectrum imaging device camera 204, proximity sensors, transmitters, bolometers, or receivers to provide feedback to the patient to aid the patient in properly aligning medical instrument 224 relative to the patient's body part of interest when performing a diagnostic measurement. A person skilled in the art will appreciate that not all sensors 208, 226, beacon, pressure, altimeter, etc., need to operate at all times. Any number of sensors may be partially or completely disabled, e.g., to conserve energy.

In embodiments, the sensor emitter comprises a light signal emitted by IR LED 226 or any other identifiable marker that may be used as a reference signal. In embodiments, the reference signal may be used to identify a location, e.g., within an image and based on a characteristic that distinguishes the reference from other parts of the image. In embodiments, the reference signal is representative of a difference between the position of medical instrument 224 and a preferred location relative to a patient's body. In embodiments, spectrum imaging device camera 204 displays, e.g., via monitor 206, the position of medical instrument 224 and the reference signal at the preferred location so as to allow the patient to determine the position of medical instrument 224 and adjust the position relative to the preferred location, displayed by spectrum imaging device camera 204.

Spectrum imaging device camera 204, proximity sensor, transmitter, receiver, bolometer, or any other suitable device may be used to locate or track the reference signal, e.g., within the image, relative to a body part of the patient. In embodiments, this may be accomplished by using an overlay method that overlays an image of a body part of the patient against an ideal model of device usage to enable real-time feedback for the patient. The reference signal along with signals from other sensors, e.g., patient-equipment activity inertial sensor 208, may be used to identify a position, location, angle, orientation, or usage associated with medical instrument 224 to monitor and guide a patient's placement of medical instrument 224 at a target location and accurately activate a device for measurement.

In embodiments, e.g., upon receipt of a request signal, microcontroller 202 activates one or more medical instruments 224 to perform measurements and sends data related to the measurement back to microcontroller 202. The measured data and other data associated with a physical condition may be automatically recorded and a usage accuracy of medical instrument 224 may be monitored.

In embodiments, microcontroller 202 uses an image in any spectrum, motion signal and/or an orientation signal by patient-equipment activity inertial sensor 208 to compensate or correct the vital signs data output by medical instrument 224. Data compensation or correction may comprise filtering out certain data as likely being corrupted by parasitic effects and erroneous readings that result from medical instrument 224 being exposed to unwanted movements caused by perturbations or, e.g., the effect of movements of the patient's target measurement body part.

In embodiments, signals from two or more medical instruments 224, or from medical instrument 224 and patient-activity activity system inertial sensor 208, are combined, for example, to reduce signal latency and increase correlation between signals to further improve the ability of vital signs measurement system 200 to reject motion artifacts to remove false readings and, therefore, enable a more accurate interpretation of the measured vital signs data.

In embodiments, spectrum imaging device camera 204 displays actual or simulated images and videos of the patient and medical instrument 224 to assist the patient in locating a desired position for medical instrument 224 when performing the measurement so as to increase measurement accuracy. Spectrum imaging device camera 204 may use image or facial recognition software to identify and display eyes, mouth, nose, ears, torso, or any other part of the patient's body as reference.

In embodiments, vital signs measurement system 200 uses machine vision software that analyzes measured image data and compares image features to features in a database, e.g., to detect an incomplete image for a target body part, to monitor the accuracy of a measurement and determine a corresponding score. In embodiments, if the score falls below a certain threshold system 200 may provide detailed guidance for improving measurement accuracy or to receive a more complete image, e.g., by providing instructions on how to change an angle or depth of an otoscope relative to the patient's ear.

In embodiments, the machine vision software may use an overlay method to mimic a patient's posture/movements to provide detailed and interactive instructions, e.g., by displaying a character, image of the patient, graphic, or avatar on monitor 206 to provide feedback to the patient. The instructions, image, or avatar may start or stop and decide what help instruction to display based on the type of medical instrument 224, the data from spectrum imaging device camera 204, patient-equipment activity sensors inertial sensors 208, bolometer, transmitter and receiver, and/or identifiable marker IR LED 226 (an image, a measured position or angle, etc.), and a comparison of the data to idealized data. This further aids the patient in correctly positioning and operating medical instrument 224 relative to the patient's body, ensures a high level of accuracy when operating medical instrument 224, and solves potential issues that the patient may encounter when using medical instrument 224.

In embodiments, instructions may be provided via monitor 206 and describe in audio/visual format and in any desired level of detail, how to use medical instrument 224 to perform a diagnostic test or measurement, e.g., how to take temperature, so as to enable patients to perform measurements of clinical-grade accuracy. In embodiments, each sensor 208, 226, e.g., proximity, bolometer, transmitter/receiver may be associated with a device usage accuracy score. A device usage accuracy score generator (not shown), which may be implemented in microcontroller 202, may use the sensor data to generate a medical instrument usage accuracy score that is representative of the reliability of medical instrument 224 measurement on the patient. In embodiments, the score may be based on a difference between an actual position of medical instrument 224 and a preferred position. In addition, the score may be based on detecting a motion, e.g., during a measurement. In embodiments, in response to determining that the accuracy score falls below a threshold, a repeat measurement or device usage assistance may be requested. In embodiments, the device usage accuracy score is derived from an error vector generated for one or more sensors 208, 226. The resulting device usage accuracy score may be used when generating or evaluating medical diagnosis data.

In embodiments, microcontroller 202 analyzes the patient measured medical instrument data to generate a trust score indicative of the acceptable range of the medical instrument. For example, by comparing the medical instrument measurement data against reference measurement data or reference measurement data that would be expected from medical instrument 224. As with device usage accuracy score, the trust score may be used when generating or evaluating a medical diagnosis data.

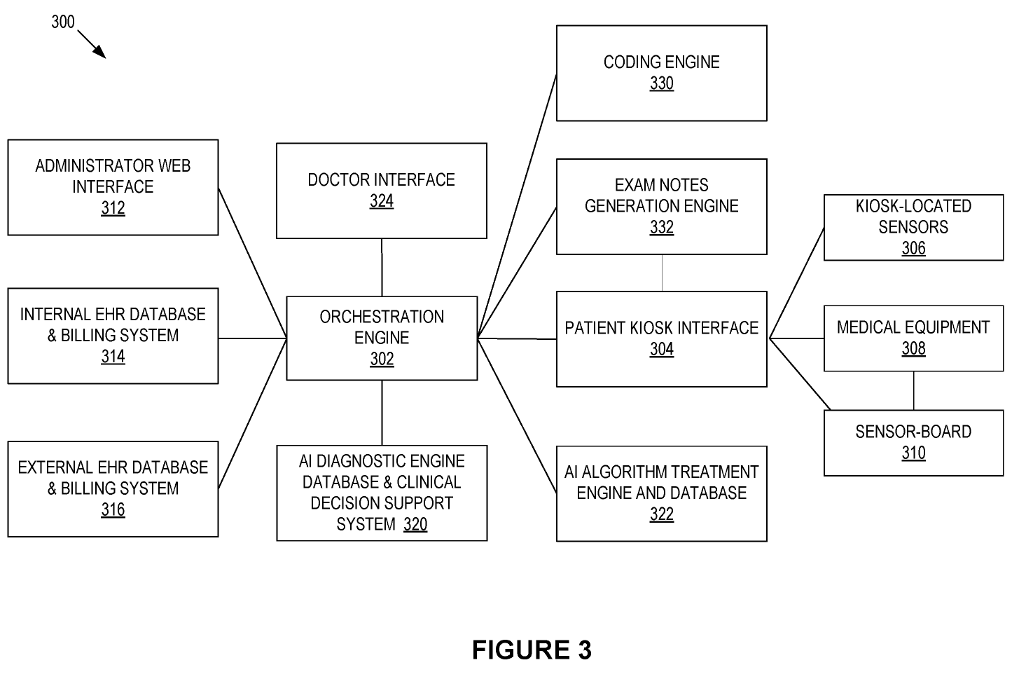

FIG. 3 illustrates an exemplary medical data system according to embodiments of the present disclosure. Medical data system 300 comprises main orchestration engine 302, patient kiosk interface 304, doctor interface 324, kiosk-located sensors 306, medical equipment 308, which may be coupled to sensor-board 310, administrator web interface 312, internal EHR database and billing system 314, external EHR database and billing system 316, Artificial Intelligence (AI) diagnostic engine and clinical decision support system 320, AI treatment engine and database 322, coding engine 330, and exam notes generation engine 332.

In operation, patient kiosk interface 304, which may be a touch, voice, or text interface that is implemented into a tablet, computer, mobile device, or any other electronic device, receives patient-identifiable information, e.g., via a terminal or kiosk.

In embodiments, the patient-identifiable information and/or matching patient-identifiable information may be retrieved from internal EHR database and billing system 314 or searched for and downloaded from external EHR database 316, which may be a public or private database such as a proprietary EHR database. In embodiments, if internal EHR database 314 and external EHR database 316 comprise no record for a patient, a new patient record may be created in internal EHR database and billing system 314. In embodiments, internal EHR database and billing system 314 is populated based on external EHR database 316.

In embodiments, data generated by medical equipment 308 is adjusted using measurement accuracy scores that are based on a position accuracy or reliability associated with using medical equipment 308, e.g., by applying weights to the diagnostic medical information so as to correct for systemic equipment errors or equipment usage errors. In embodiments, generating the diagnostic data comprises adjusting diagnostic probabilities based on the device accuracy scores or a patient trust score associated with a patient. The patient trust score may be based on a patient response or the relative accuracy of responses by other patients. Orchestration engine 302 may record a patient's interactions with patient-kiosk interface 304 and other actions by a patient in diagnostic EHR database and billing system 314 or external EHR database and billing system 316.

In embodiments, orchestration engine 302 coordinates gathering data from the AI diagnostic engine and clinical decision support system 320 which links gathered data to symptoms, diagnostics, and AI treatment engine and database 322 to automatically generate codes for tracking, diagnostic, follow-up, and billing purposes. Codes may comprise medical examination codes, treatment codes, procedural codes (e.g., numbers or alphanumeric codes that may be used to identify specific health interventions taken by medical professionals, such as ICPM, ICHI, and other codes that may describe a level of medical exam), lab codes, medical image codes, billing codes, pharmaceutical codes (e.g., ATC, NDC, and other codes that identify medications), and topographical codes that indicate a specific location in the body, e.g., ICD-O or SNOMED.

In embodiments, system 300 may comprise a diagnostic generation engine (not shown in FIG. 3) that may, for example, be implemented into coding engine 330. The diagnostic generation engine may generate diagnostic codes e.g., ICD-9-CM, ICD-10, which may be used to identify diseases, disorders, and symptoms. In embodiments, diagnostic codes maybe used to determine morbidity and mortality.

In embodiments, generated codes may be used when generating a visit report and to update internal EHR database and billing system 314 and/or external EHR database and billing system 316. In embodiments, the visit report is exported, e.g., by using a HIPAA compliant method, to external EHR database and billing system 316. It is understood that while generated codes may follow any type of coding system and guidelines, rules, or regulations (e.g., AMA billing guidelines) specific to health care, other codes may equally be generated.

In embodiments, exam notes generation engine 332 aids a doctor in writing a report, such as a medical examination notes that may be used in a medical evaluation report. In embodiments, exam notes generation engine 332, for example in concert with AI diagnostic engine and clinical decision support system 320 and/or AI treatment engine and database 322, generates a text description of exam associated with the patient's use of system 300, patient-doctor interaction, and diagnosis or treatment decisions resulting therefrom. Exam notes may comprise any type of information related to the patient, items examined by system 300 or a doctor, diagnosis justification, treatment option justification, and other conclusions. As an example, exam notes generation engine 332 may at least partially populate a text with notes related to patient information, diagnostic medical information, and other diagnosis or treatment options and a degree of their relevance (e.g., numerical/probabilistic data that supports a justification why a specific illness or treatment option was not selected).

In embodiments, notes comprise patient-related data such as patient interaction information that was used to make a recommendation regarding a particular diagnosis or treatment. It is understood that the amount and detail of notes may reflect the closeness of a decision and the severity of an illness. Doctor notes for treatment may comprise questions that a patient was asked regarding treatment and reasons for choosing a particular treatment and not choosing alternative treatments provided by AI treatment engine and database 322. In embodiments, provider notes may be included in a patient visit report and also be exported to populate an internal or external EHR database and billing system.

In embodiments, orchestration engine 302 forwards a request for patient-related data to patient kiosk interface 304 to solicit patient input either directly from a patient, assistant, or medical professional in the form of answers to questions or from a medical equipment measurement (e.g., vital signs). The patient-related data may be received via secure communication and stored in internal EHR database and billing system 314. It is understood that patient input may include confirmation information, e.g., for a returning patient.

In embodiments, based on an AI diagnostic algorithm, orchestration engine 302 may identify potential illnesses and request additional medical and/or non-medical (e.g., sensor) data that may be used to narrow down the number of potential illnesses and generate diagnostic probability results.

In embodiments, the diagnostic result is generated by using, for example, data from kiosk-located sensors 306, medical equipment 308, sensor board 310, patient history, demographics, and preexisting conditions stored in either the internal EHR 314 or external EHR database 316. In embodiments, orchestration engine 302, AI diagnostic engine and clinical decision support system 320, or a dedicated exam level generation engine (not shown in FIG. 3.) suggests a level of exam in association with illness risk profile and commonly accepted physical exam guidelines. Exam level, actual exams performed, illness probability, and other information may be used for provider interface checklists that meet regulatory requirements and to generate billing codes.

In embodiments, AI diagnostic engine and clinical decision support system 320, may generate a criticality score representative of probability that a particular patient will suffer a negative health effect for a period of time from non-treatment may be assigned to an illness or a combination of illnesses as related to a specific patient situation. Criticality scores may be generated by taking into account patients' demographic, medical history, and any other patient-related information. Criticality scores may be correlated to each other and normalized and, in embodiments, may be used when calculating a malpractice score for the patient. A criticality score of 1, for example, may indicate that no long-lasting negative health effect is expected from an identified illness, while a criticality score of 10 indicates that that the patient may prematurely die if the patient in fact has that illness, but the illness remains untreated.

As an example, a mid-aged patient of general good health and having weight, blood pressure, and BMI data indicating a normal or healthy range is not likely to suffer long-lasting negative health effects from non-treatment of a common cold. As a result, a criticality score of 1 may be assigned to the common cold for this patient. Similarly, the previously referred to patient is likely to suffer moderately negative health effects from non-treatment of strep throat, such that a criticality score of 4 may be assigned for strep throat. For the same patient, the identified illness of pneumonia may be assigned a criticality score of 6, and for lung cancer, the criticality score may be 9.

In embodiments, AI treatment engine and database 322 receives from AI diagnostic engine and clinical decision support system 320 diagnosis and patient-related data, e.g., in the form of a list of potential illnesses and their probabilities together with other information about a patient. Based on the diagnosis and patient-related data, AI treatment engine and database 322 may determine treatment recommendations/suggestions/options for each of the potential illness. Treatment options may comprise automatically selected medicines, instructions to the patient regarding a level of physical activities, nutrition, or other instruction, and exam requests, such as suggested patient treatment actions, lab tests, and imaging requests that may be shared with a doctor.

In embodiments, AI treatment engine and database 322 generates for a patient a customized treatment plan, for example, based on an analysis of likelihoods of success for each of a set of treatment options, taking into consideration factors such as possible drug interactions and side-effects (e.g., whether the patient is allergic to certain medicines), patient demographics, and the like. In embodiments, the treatment plan comprises instructions to the patient, requests for medical images and lab reports, and suggestion regarding medication. In embodiments, treatment options are ranked according to the likelihood of success for a particular patient.

In embodiments, AI treatment engine and database 322 is communicatively coupled to a provider application (e.g., on a tablet) to exchange treatment information with a doctor. The provider application may be used to provide the doctor with additional details on diagnosis and treatment, such as information regarding how a probability for each illness or condition is calculated and based on which symptom(s) or why certain medication was chosen as the best treatment option.

For example, once a doctor accepts a patient, a monitor may display the output of AI diagnostic engine and clinical decision support system 320 as showing a 60% probability that the patient has the common cold, 15% probability that the patient has strep throat, 15% probability that the patient has an ear infection, 5% probability that the patient has pneumonia, and a 5% probability that the patient has lung cancer. In embodiments, the probabilities are normalized, such that their sum equals 100% or some other measure that relates to the provider.

In embodiments, the doctor may select items on a monitor to access additional details such as images, measurement data, and any other patient-related information, e.g., to evaluate a treatment plan proposed by AI treatment engine and database 322.

In embodiments, AI treatment engine and database 322 may determine the risk of false negative for each illness (e.g., strep, pneumonia, and lung cancer), even of a doctor determined that the patient has only one illness, and initiate test/lab requests, e.g., a throat swab for strep, and an x-ray for pneumonia/lung cancer that may be ordered automatically or with doctor approval (e.g., per AMA guidelines).

In embodiments, AI treatment engine and database 322 combines gathered data with symptom, diagnostic, and treatment information and generates any type of diagnostic, physical exam level, and treatment codes, for example, for billing purposes or to satisfy regulatory requirements and/or physician guidelines, billing, and continued treatment purposes. In embodiments, treatment codes are based on drug information, a patient preference, a patient medical history, and the like.

In embodiments, codes, doctor notes, and a list of additional exam requirements may be included in a visit report and used to automatically update internal EHR database and billing system 314 or external EHR database and billing system 316. The visit report may then be exported, e.g., by a HIPAA compliant method, to external EHR database and billing system 316.

In embodiments, AI treatment engine and database 322 estimates when a particular treatment is predicted to show health improvements for the patient. For example, if a predicted time period during which a patient diagnosed with strep throat and receiving antibiotics should start feeling better is three days, then, on the third day, medical data system 300 may initiate a notice to the patient to solicit patient feedback to determine whether the patient's health condition, in fact, has improved as predicted.

It is understood that patient may be asked to provide feedback at any time and that feedback may comprise any type of inquiries related to the patient's condition. In embodiments, the inquiry comprises questions regarding the patient's perceived accuracy of the diagnosis and/or treatment. In embodiments, if the patient does not affirm the prediction, e.g., because the symptoms are worsening or the diagnoses was incorrect, the patient may be granted continued access to system 300 for a renewed diagnosis.

In embodiments, any part of the information gathered by medical data system 300 may be utilized to improve machine learning processes of AI diagnostic engine and clinical decision support system 320 and AI treatment engine and database 322 and, thus, improve diagnoses and treatment capabilities of system 300.

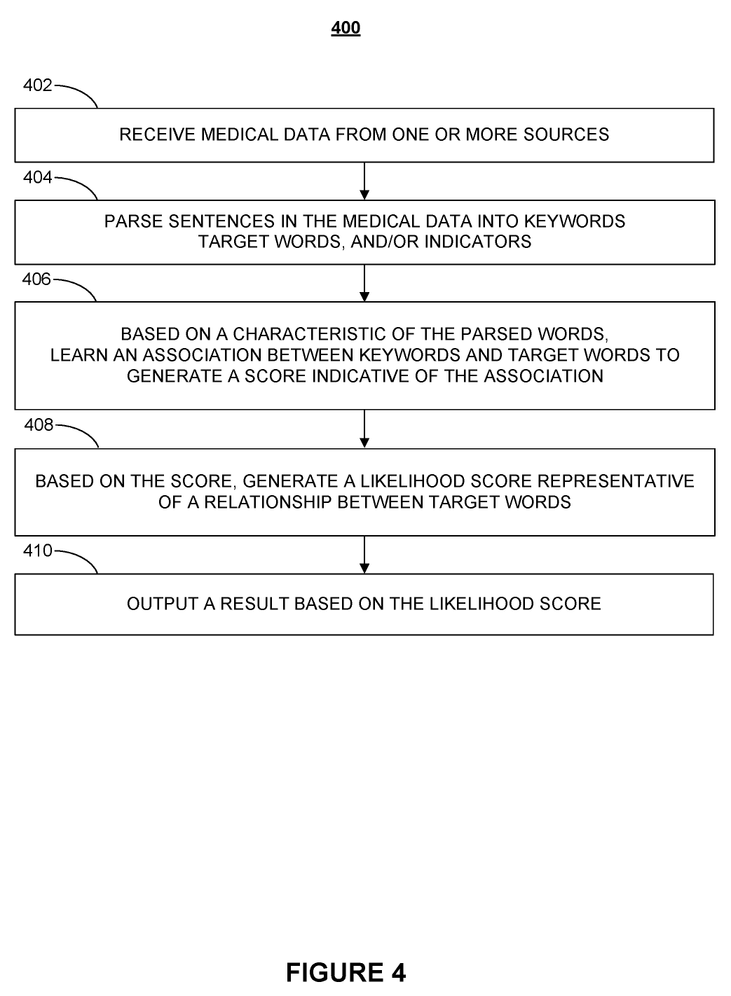

FIG. 4 is a flowchart illustrating a process for training a medical data model according to various embodiments of the present disclosure. Process 400 begins at step 402, when raw or processed medical data and data related to medical data is obtained from sources such as electronic and non-electronic publications, medical records, transcripts, medical literature (e.g., journal articles), electronic databases, etc. It is understood that the medical data may have any suitable format and may be received in any type of medium (e.g., images, voice recordings, etc.).

At step 404, in embodiments, using a parsing method, e.g., natural language programing (NLP), sentences contained in the body of data may be queried and parsed into terms and/or phrases, e.g., keywords and target words. In embodiments, keywords may comprise modifiers, such as quantifiers (e.g., “rare,” “occasional,” “four in a thousand,” etc.); qualifiers (“severe,” “thick,” “elevated,” etc.); and indicators, such as “smoking” that may be a viewed as factors and/or events, that the medical data model may use, for example, in diagnosing an illness. In embodiments, target words may be categorized into a number of categories, such as symptoms, illnesses, treatments, outcomes to generate a correlation matrix that comprises the categories.

To overcome the limitations of language, in embodiments, a semantic model identifies and treats similarly used terminology (i.e., terms that may have comparable meaning) as interchangeable, e.g., by categorizing related terms and phrases. In embodiments, classified related terms may be generalized, e.g., by selecting a term and applying it to an identified category, in effect, treating the selected term as having a generalized meaning. Conversely, in embodiments, the semantic model may use a parsing method to treat a single term that may have several meanings differently, e.g., based on context, proximity to other terms, or any other characteristic.

In embodiments, a keyword or modifier may be weighed based on context. For example, the word “rare” may be weighed differently based on nearby word or phrases that identify a sample size, e.g., a population size used in a research paper, which mentions the rarity of a given illness.

In embodiments, phrases, terms, or even partial terms may be assigned a definition, e.g., a concept, that advantageously creates context and allows to understand relationships, e.g., when searching for or comparing related words or items. In embodiments, any partial term (e.g., a Latin root or a base root) of a given parsed term may be stored and retrieved from a database.

It is understood that while parsing is discussed in the context of words and sentences herein, such parsing methods and, in fact, any combination of known technologies and tools (e.g., neural network analysis, statistical analysis, and machine learning) may equally be applied to analyze and interpret the full breadth of other medical data “language,” such as images and sounds. It is further understood different types of data may be parsed in different ways.

At step 406, e.g., based on a relationship between the parsed terms, such as a position of words within a sentence or any other characteristic of parsed words, keywords, target words, and/or indicators may be identified and, in embodiments, associated with each other to generate scores that quantify the relationship, closeness, or other attributes. For example, the phrases “high temperature” or “elevated body temperature” may be categorized as “fever.” Each term may be categorized based on the closeness of the match between a word and a related word. In embodiments, identifying the keywords and/or target words comprises learning, in a training phase, a relationship between keywords, target words, and indicators.

In embodiments, based on the generated score, at step 408, a likelihood score that is representative of a relationship between one or more target words (e.g., symptoms, illnesses, treatments, outcomes) is generated. For example, a causal relationship between a first target word, such as a symptom, and a second target word, such as an illness.

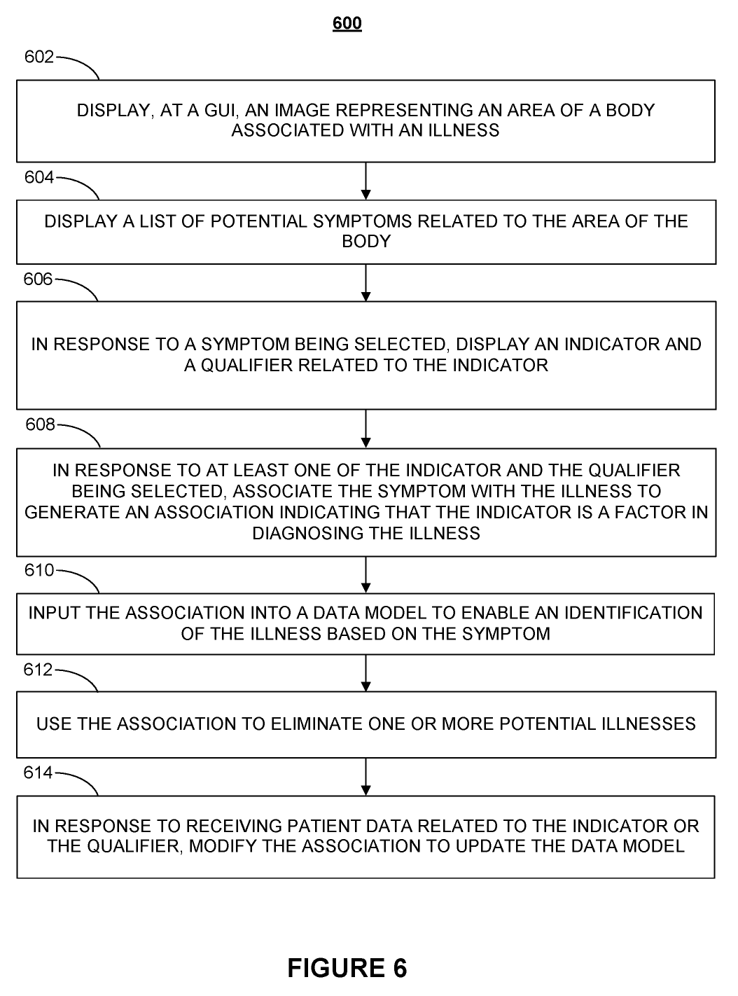

In embodiments, a relationship may be established in response to a medical professional making a selection related to a list of potential symptoms or directly matching, e.g., a symptom to an illness. In embodiments, the selection may involve displaying any number of keywords and indicators related to the target word. As further discussed with respect to FIG. 6, the selection may further involve displaying for a given for the illness, e.g., on a monitor, an image that represents an area of a body.

Finally, at step 410, a result that, in embodiments, is representative of the likelihood score may be output. In embodiments, outputting the result may comprise eliminating one or more potential illnesses or symptoms, e.g., based on the likelihood score. In embodiments, the likelihood score may be converted into a probability or percentage that may be output as a result of a predictive engine.

It is understood that outputting the result may further comprise visualization techniques to present analysis results. It is further understood that any input or output may be reviewed and adjusted, e.g., by using input from a physician. For example, calculated likelihoods may be manipulated by a medical professional based on personal knowledge or interpretation of medical and non-medical data, e.g., patient-specific data.

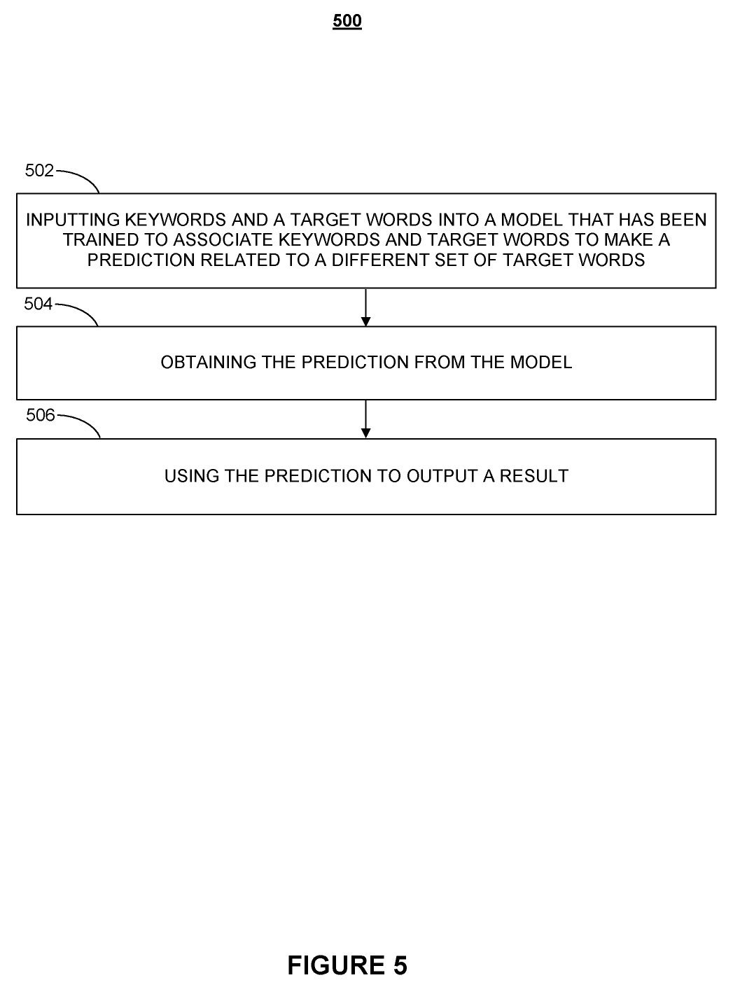

FIG. 5 is a flowchart illustrating a process for using a medical data model, according to various embodiments of the present disclosure, to make medical predictions. Process 500 begins at step 502, when a number of keywords and target words is input into a medical data model. In embodiments, at least one target word comprises a symptom, and another target word comprises an illness. In embodiments, the data model has been trained to associate the keywords and at least one target word to make a prediction that is related to a number of target words. In embodiments, making the prediction comprises eliminating one or more potential illnesses based on the likelihood or a likelihood score.

In embodiments, one or more keywords comprise a modifier, measure, and/or an indicator. The measure may comprise weight data, a range, an option, a frequency, a percentage, a likelihood, and so on.

The indicator may be a negative indicator and, in embodiments, comprise information related to a pain descriptor, an immunization, an allergy, a travel risk, an alcohol use, an occupational risk, a diet, a pet risk, a food source, a physical condition, a neurological condition, past patient medical data, etc.

At step 504, the prediction is obtained from the model and used, at step 506, to output a result that, in embodiments comprises an identification of an illness based on a number of symptoms, treatments, and/or outcomes. An outcome may be, for example, the result or success of a treatment given a set of symptoms and illnesses. A known drug or type of drug may have a known outcome or outcome probability that at least in part depends on medical information, such as patient-specific information (e.g., a patient demographic) and other information (e.g., number and types of symptoms). As an example, a patient from a certain demographic having a number of certain symptoms may respond to a particular treatment differently that a patient from a different demographic or a patient a different set of symptoms.

In embodiments, this outcome-related information may be provided to the system by feeding follow-up data back into the system. For example, information indicating how a patient or a group of patients responds to a particular treatment may be obtained at various stages of treatment and may be provided to the system for analysis and/or incorporation into a machine learning model that learns the effects of certain treatments on patients. This allows the system to predict the accuracy of a given treatment on a given type of patient before predicting or suggesting a customized treatment that may achieve an individualized, effective outcome.

Although outcome-related information is discussed herein with reference to treatment information, it is understood that outcome-related information may comprise any type of medical information, such as symptom and illness information. It is further understood that there is no limitation on the granularity or the type of information that may comprise timing data. For example, the effectiveness of a medication may be measured in stages over a period of time, such that each stage can be associated with a predicted outcome.

FIG. 6 is a flowchart illustrating a process for using expert knowledge to create and/or train a medical data model according to various embodiments of the present disclosure. Process 600 begins at step 602, when for a given illness an image representing an area of a human body is displayed, e.g., at a GUI. It is understood that illustrations comprising any desired level of detail about a region may also be displayed. As used herein, the term “display” relates to any form of audio or visual presentation of potential symptoms, indicators, measures, and the like, having any known format, e.g., in form of a drop-down menu.

In embodiments, one or more regions of a body may be displayed, for example, in a left-hand side column of a screen design and, in a right-hand side column, data related to the shown body region(s) may be displayed. In embodiments, the column to the left may be used to locate the exact location or specific area of the body, while the column to the right may be a tabular representation of specific symptoms and any type of data (e.g., pictures, lists, etc.) associated with the displayed body region. The interaction with images, advantageously, facilitates a more efficient and quicker path to identify a specific body location than textual, tabular navigation. Nevertheless, one or more functions may have the same result for both columns.

In embodiments, for a given symptom, a doctor may assign different confidence levels to different regions of the body. In embodiments, available confidence values may be distributed as follows: n/a—0%, low—25%, medium—50%, high—75%, and probable—100%. For example, if a symptom is typically associated with a particular area of the body, that area may be assigned a 100% confidence level. As a result, once a patient selects that particular body region, a diagnostic engine may assign a higher confidence level to some or all related or auxiliary information (e.g., pain level) provided by the patient.

On the other hand, a doctor may assign lower confidence levels (e.g., 50%) to other areas that are less likely affected by the symptom(s), such that once a patient selects an area associated with a lower confidence level, a diagnostic engine may assign a relatively lower confidence level to information provided by the patient that is related to that particular patient-selected area.

As used herein, an indicator may comprise information related to one or more related or unrelated factors, such as an immunization, an allergy, a time of day or duration, a travel risk, a substance usage, a physical activity level, an occupational risk, a predisposition, an emotional condition, dietary factors, exposure to certain pets, foods, chemicals, or environmental factors, such as plants, extreme temperatures, etc., a weight change, a physical condition, a medical condition, a genetic condition, and a neurological condition. In embodiments, an indicator may comprise past patient medical data, including a pain descriptor.

In embodiments, a measure or information relevant to an indicator may be used to quantify and compare patient data, e.g., other patients. As used herein, a measure may comprise numerical data, a timing information, and/or at least one of a weight data, a range, an option, a frequency, a percentage, and a likelihood.

In embodiments, a doctor may associate a symptom, indicator, or measure with an illness by assigning a likelihood indicative of the presence of the symptom, indicator, or measure being a possible indicator of the presence of the illness, or at the least a possible relationship between the illness and the symptom, indicator, or measure. The practitioner may, for example, assign a likelihood by selecting among choices, such as “somewhat likely,” “likely,” “very likely,” “definitely.” However, this is not intended as a limitation on the scope of the invention, as any other method, e.g., numerical choices, may equally be used and implemented to create a level of association.

For example, if a doctor elects to use a time element as indicator to associate the time element with a symptom, e.g., by identifying time elements that are relevant for a symptom, this may be accomplished by making selections that correspond to a given timeline. Assuming a timeline of 12 weeks is to be considered, then a choice of 5 selection buttons may be presented in a menu (e.g., for 0-3 weeks, 4-6 weeks, 7-9 weeks, 10-12 weeks, and more than 12 weeks). The doctor may choose the first three buttons to reflect that if a relevant symptom has appeared in the last 9 weeks, a relatively higher weighting should be given to the symptom than otherwise.

In embodiments, the identified time element may reflect a trend, e.g., that a particular symptom is increasing or decreasing, or the time element may be used to help differentiate an importance in the time line, e.g., a symptom may be more critical if it occurred more recently than if it has been present for several weeks or months.

In embodiments, the presence and importance of a symptom may be linked to descriptive indicators that may be implemented by buttons that when activated may help match specifics of an illness to patient feedback, e.g., description of a pain level.

In embodiments, the doctor may select an indicator (e.g., alcohol use) and assign to that indicator a qualifier or measure that represents a likelihood of the indicator being a relevant factor in diagnosing a particular illness. As an example, an indicator may be a range of alcoholic beverages that a patient typically consumes in a given time period that the doctor believes would be a relevant factor in a diagnosis of a certain illness. In embodiments, the indicator may be assigned a relationship, e.g., via a proportionally factor that causes proportionally more weight to be assigned to the indicator.

In embodiments, the qualifier or measure may be a frequency (e.g., a frequency of tobacco use) selected from a range of frequencies. In embodiments, the qualifier or measure is determined by the doctor. For example, by setting the possible range of frequencies of tobacco use to the number of tobacco products consumed per day (e.g., a range from zero to 100), such that a question related to tobacco use may display, e.g., in a drop down menu on a scale from zero tobacco products consumed per day to 100 tobacco products consumed per day.

In embodiments, the doctor may create a relationship, e.g., between ranges of two different indicators, to indicate that both indicators should be weighed according to the relationship when determining each individual indicators' effect on a given illness.

In embodiments, the doctor may assign different factors to different ranges. Assuming that a patient physical activity level is used as a factor in diagnosing an illness, the doctor may choose a set of possible ranges of physical activity level, e.g., on a scale of 0 to 100, such as 0-20, 21-40, 41-60, 61-80, and 81-100, with 0 representing no physical activity. In embodiments, the doctor may choose the set of possible ranges from a menu, modify ranges in and existing menu, or newly define a range and sub-range therein. Other option may include yes/no questions, relative comparisons with known prior conditions, etc.

In embodiments, due to variances in how doctors may describe the same condition, symptom, and illness, selections (e.g., identified symptoms) by different doctors may be treated differently. For example, in embodiments, a description by a particular doctor may be differently weighed than the description by other doctors.

It is understood that data may be represented and associated with any desired level of depth. In embodiments, the level of choices may be adjusted based on the type of a symptom, illness, other any characteristic related thereto.

Certain symptoms may be weighed more heavily than others. It is understood, that while herein symptoms are associated with a given illness, in embodiments, illnesses may be associated with a given symptom as part of a process that identifies an illness based on any number of symptoms.

At step 604, in response to an area being selected, a list of potential symptoms related to the area of the body may be displayed. For example, once a body area has been identified as relevant for an illness a doctor may be prompted to select from a drop-down box one or more potential symptoms associated with the area that can be mapped onto one or more illnesses. In embodiments, selection requires affirmative action to indicate that a certain choice does not apply. For example, a check box for an option in a decision tool may have to be selected to indicate that the option does not apply. This aids in reducing the risk that an option has been overlooked or not considered, in effect, forcing decisions regarding a predetermined number of areas, e.g., when defining an illness.

It is understood that any known means of navigation may be used to enable convenient and quick data selection, data entry, and data association. For example, data entry may be enabled by any method related to reducing keyboard interaction and variability to simplify entering data, selecting between options, and answering questions.

At step 606, in response to a symptom being selected, an indicator and a qualifier related to that indicator may be displayed. It is understood that the indicator may be a negative indicator that represents the absence of a condition. In embodiments, the presence (or lack) of a symptom may be assigned a positive or negative indicator for a given illness. For example, the presence of visible spots on a person's skin may be assigned a positive indicator for chicken pox; contrariwise, the same spots may be assigned a negative indicator for an illness such as roseola.

At step 608, in embodiments, in response to the indicator or qualifier being selected, a symptom that has been selected at step 606 may be associated with the illness to generate an association indicating that indicator is a factor in diagnosing the illness.

At step 610, the association is input to a data model to enable an identification of the illness based on the symptom.

At step 612, the association is used to eliminate one or more potential illnesses. In embodiments, the associations may be used in the data model as a decision tool that is capable of distinguishing between illnesses. In particular, the decision tool may distinguish between small variances in illnesses, e.g., different types of diabetes.

In embodiments, the doctor-provided likelihoods of associations may be used to train a predictive engine, e.g., a supervised machine learning tool that employs a machine learning algorithm to predict an outcome, such as an illness based on a given set of symptoms as inputs. In embodiments, the predictive engine may use additional training data, such as a number of occurrences, locations, and other entries, in order to define the quality of/calculate likelihood scores to predict an outcome. In embodiments, at least some likelihoods of associations, e.g., between illnesses, symptoms, treatments, and/or outcomes, are generated by using a method for creating a data model discussed with respect to FIG. 4 above. In other words, there is no limitation on the source and type of information that may be used, learned, analyzed, and updated—whether with or without the aid of a medical professional—to extract medical information that serves to make medical predictions.

At step 614, in response to receiving patient data related to the indicator or qualifier the association is modified to update the data model. In embodiments, descriptors, e.g., from patient feedback provided along with location data, may be matched as relevant to one or more illnesses. Feedback may be collected from actual users of the system to improve the predictive value of the system in associating illnesses with corresponding symptoms and vice versa.

In embodiments, the doctor may assign to a low physical activity range a value that is different than for a high activity range, such that a patient identified as inactive may be considered a more likely candidate to whom a specific illness applies. In embodiments, where no qualifier or measure exists, the doctor may create a suitable qualifier or measure and appropriate unit.

In embodiments, the doctor may select a type and timing of a past event, e.g., a prior immunization, as a factor in diagnosing an illness. In embodiments, the time passed since the event may be indicated on a time scale. A range may indicate that a relatively recent immunization within a certain time frame would be weighed higher than a less recent immunization.

In embodiments, the doctor may be asked to assign a confidence level, e.g., to a region of a body that is commonly misidentified by patients as being the source of pain. The confidence level may be reflective of a likelihood that a patient will fail to accurately identify or select a specific symptom or body part.

In embodiments, a doctor selects factors (e.g., time, inaction/action, body stance) that provide relief or cause a change (improvement or worsening) of a condition/pain location/sensation/symptom, etc.

In embodiments, a time maybe calculated at which the selected treatment is expected to show a result and patient feedback may be requested, e.g., as part of a feedback process, to improve diagnosis and treatment reliability. In embodiments, the selected treatment and/or the patient feedback may be used to adjusting one or more of the metrics. For example, based on one or more of the metrics a series of treatment plans maybe generated by using an algorithm that combines the metrics. In embodiments, calculating the selected treatment comprises using cost as a factor.

One skilled in the art will recognize that: (1) certain steps may optionally be performed; (2) steps may not be limited to the specific order set forth herein; and (3) certain steps may be performed in different orders; and (4) certain steps may be done concurrently.

In embodiments, one or more computing systems, such as mobile/tablet/computer or the automated diagnostic system, may be configured to perform one or more of the methods, functions, and/or operations presented herein. Systems that implement at least one or more of the methods, functions, and/or operations described herein may comprise an application or applications operating on at least one computing system. The computing system may comprise one or more computers and one or more databases. The computer system may be a single system, a distributed system, a cloud-based computer system, or a combination thereof.

It shall be noted that the present disclosure may be implemented in any instruction-execution/computing device or system capable of processing data, including, without limitation phones, laptop computers, desktop computers, and servers. The present disclosure may also be implemented into other computing devices and systems. Furthermore, aspects of the present disclosure may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

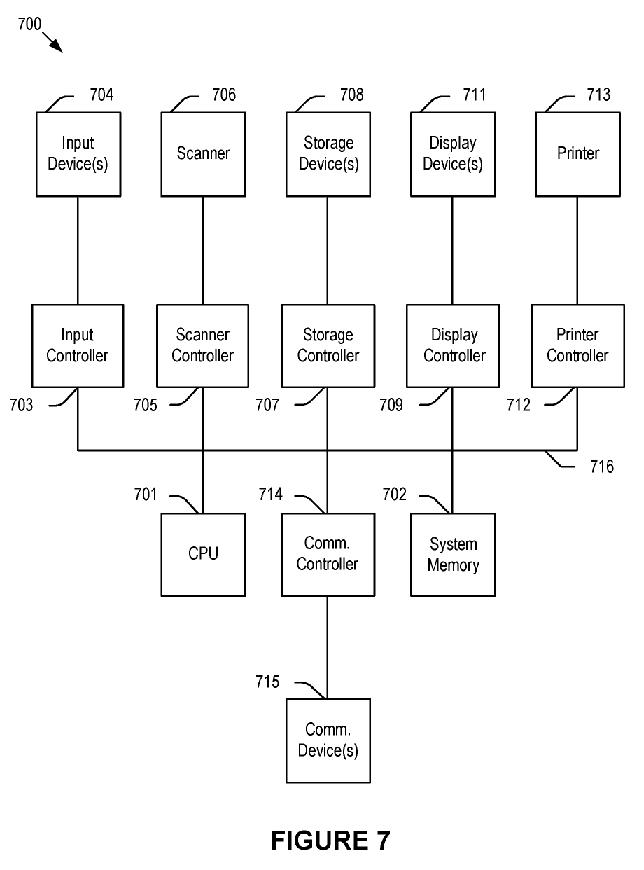

Having described the details of the disclosure, an exemplary system that may be used to implement one or more aspects of the present disclosure is described next with reference to FIG. 7. Each of patient interface station 107 and automated diagnostic system 102 in FIG. 1 may comprise one or more components in the system 700. As illustrated in FIG. 7, system 700 includes a central processing unit (CPU) 701 that provides computing resources and controls the computer. CPU 701 may be implemented with a microprocessor or the like, and may also include a graphics processor and/or a floating point coprocessor for mathematical computations. System 700 may also include a system memory 702, which may be in the form of random-access memory (RAM) and read-only memory (ROM).

A number of controllers and peripheral devices may also be provided, as shown in FIG. 7. An input controller 703 represents an interface to various input device(s) 704, such as a keyboard, mouse, or stylus. There may also be a scanner controller 705, which communicates with a scanner 706. System 700 may also include a storage controller 707 for interfacing with one or more storage devices 708 each of which includes a storage medium such as magnetic tape or disk, or an optical medium that might be used to record programs of instructions for operating systems, utilities and applications which may include embodiments of programs that implement various aspects of the present disclosure. Storage device(s) 708 may also be used to store processed data or data to be processed in accordance with the disclosure. System 700 may also include a display controller 709 for providing an interface to a display device 711, which may be a cathode ray tube (CRT), a thin film transistor (TFT) display, or other type of display. System 700 may also include a printer controller 712 for communicating with a printer 713. A communications controller 714 may interface with one or more communication devices 715, which enables system 700 to connect to remote devices through any of a variety of networks including the Internet, an Ethernet cloud, an FCoE/DCB cloud, a local area network (LAN), a wide area network (WAN), a storage area network (SAN) or through any suitable electromagnetic carrier signals including infrared signals.

In the illustrated system, all major system components may connect to a bus 716, which may represent more than one physical bus. However, various system components may or may not be in physical proximity to one another. For example, input data and/or output data may be remotely transmitted from one physical location to another. In addition, programs that implement various aspects of this disclosure may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

For purposes of this disclosure, an information handling system may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, an information handling system may be a personal computer (e.g., desktop or laptop), tablet computer, mobile device (e.g., personal digital assistant (PDA) or smart phone), server (e.g., blade server or rack server), a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, touchscreen and/or a video display. The information handling system may also include one or more buses operable to transmit communications between the various hardware components.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure.

What is claimed is:

1. A method for training a medical data model, the method comprising:
receiving medical data comprising sentences from one or more sources;
parsing the sentences to generate parsed words that comprise keywords and target words;
based on a characteristic of the parsed words, learning an association between a keyword and a first target word;
generating a score indicative of the association;
based on the score, generating a likelihood score that is representative of a relationship between, at least, the first target word and a second target word; and
outputting a result that is representative of the likelihood score.

2. The method according to claim 1, wherein the keyword comprises a modifier, and at least one of the first target word and the second target word comprises at least one of a symptom, an illness, a treatment, and an outcome.

3. The method according to claim 2, wherein the modifier comprises at least one of a qualifier and a quantifier.

4. The method according to claim 1, wherein outputting the result further comprises, based on the likelihood score, eliminating one or more potential illnesses.

5. The method according to claim 1, wherein the relationship between the first target word and the second target word is established in response to the second target word being selected from a list of potential illnesses.

6. The method according to claim 1, wherein the relationship between the first target word and the second target word is established in response to the first target word being selected from a list of potential symptoms.

7. The method according to claim 6, further comprising:
for the second target word that represents an illness, displaying on a monitor a first image that represents an area of a body;
in response to the area being selected, displaying the list of potential symptoms, the potential symptoms being related to the area of the body;
in response to a symptom being selected, displaying an indicator and a measure related to the indicator; and
and associating the symptom with the second target word to generate an association that indicates that the indicator is a factor in diagnosing the illness.

8. The method according to claim 7, further comprising inputting the association into a data model, the association enabling an identification of the illness based on the symptom.

9. The method according to claim 7, wherein displaying the first image comprises displaying a second image that comprises greater detail about the first area than the first image.

10. A method for using a medical data model to make medical predictions, the method comprising:
inputting one or more keywords and a first set of target words into a model, the model having been trained to associate the one or more keywords and at least the first set of target words to make a prediction related to a second set of target words;
obtaining the prediction from the model; and
using the prediction to output a result.

11. The method according to claim 10, wherein the one or more keywords comprise a modifier, and at least one of the first set of target words and the second set of target words comprises at least one of a symptom, an illness, a treatment, and an outcome.

12. The method according to claim 10, wherein the result comprises an identification of an illness based on a set of symptoms.

13. The method according to claim 12, wherein making the prediction comprises, based on a likelihood score, eliminating one or more potential illnesses.

14. The method according to claim 10, wherein the one or more keywords comprise an indicator.

15. The method according to claim 14, wherein the indicator comprises at least one of a pain descriptor and a negative indicator.

16. The method according to claim 14, wherein the indicator comprises information related to at least one of an immunization, an allergy, a travel risk, an alcohol use, an occupational risk, a diet, a pet risk, a food source, a physical condition, and a neurological condition.

17. The method according to claim 14, wherein the indicator comprises past patient medical data.

18. The method according to claim 10, wherein the one or more keywords comprise a measure.

19. The method according to claim 18, wherein the measure comprises timing information.

20. The method according to claim 18, wherein at least one of the result and the measure comprise at least one of weight data, a range, an option, a frequency, a percentage, and a likelihood.
